(19) United States
(12) Patent Application Publication
(10) Pub. No.: US 2004/0162467 A1
(43) Pub. Date: Aug. 19, 2004

(54) Non-invasive transudate extraction

(76) Inventor: Christian John Cook, Ragland (NZ)

Correspondence Address: JACOBSON HOLMAN PLLC, 400 SEVENTH STREET N.W., SUITE 600, WASHINGTON, DC, 20004, US

(21) Appl. No.: 10/480,234

(22) PCT Filed: Jun. 14, 2002
(86) PCT No.: PCT/NZ02/00110

(30) Foreign Application Priority Data
Jun. 14, 2001 (NZ) 512367
Dec. 14, 2001 (NZ) 516155

(51) Int. Cl. A61B 5/00

(52) U.S. Cl. 600/309; 604/22

(57) ABSTRACT

A transudate extraction device (1) for non-invasive extraction of transudate through a target area of a skin barrier of an organism subjected to sufficient ultrasonic energy from a generator (2) to induce transudation therethrough, the device including at least: a contacting means (3) for contacting said target area to receive transudate therefrom, a transudate collection chamber (6) in open communication with said contacting means to receive transudate therefrom, circulating means for circulating transudate within said collection chamber by, for example having a fluid flow (12) through an inlet port (8) and out an outlet port (9) to thereby create and maintain a concentration gradient through the skin of a said test subject, said collection chamber having a discharge opening (9) through which exudate collected therein can be discharged.

### FIELD OF THE INVENTION

This invention relates to non-invasive transudate extraction across a skin barrier in an organism.

### BACKGROUND TO THE INVENTION

It is often necessary to extract fluid through a skin barrier in order to analyse or process substances of interest in the fluid from an organism. Traditionally, extraction of fluid samples from an organism has been achieved by invasive techniques such as puncturing the skin with a hollow needle, or by making an incision with a scalpel or other instrument to allow fluid release. However, such invasive techniques have disadvantages in that they cause tissue damage. Needles can also suffer from the disadvantage that they become clogged or may not provide a representative sample because, by their nature, they extract from a very localised site. They also often cause physical pain and, in some cases, mental fear in a patient.

In the case of some analytes, intensive repeat sampling over a short time range is needed. Removal of blood by invasive means is problematic under these conditions.

A number of non-invasive techniques have been developed to avoid the use of needles and other invasive techniques. These include, flash heating of skin by light radiation, electroporation and ultrasound. The flash heating of tissue causes tissue damage and the release of analyte from these tissues may not represent a true reading of the actual analyte level in the tissues concerned. Flash heating also may involve unacceptable levels of radiation. Electroporation may cause local tissue damage and may form undesirable long-term channels in tissue whereby bodily fluids can seep out through these channels. Undesirable foreign substances may also inadvertently be introduced into an organism through such channels.

Saliva can be non-invasively extracted. Saliva equilibrates reasonably well with many blood components, but the partitioning between the two body fluids is complex and still poorly understood. However, it appears that there are difficulties in using saliva in dynamic (nonrest) longitudinal endocrine studies. The lag time in partitioning between blood and saliva is not linear, peaks appear to be ‘averaged’ out in saliva as seen in falling recovery percentages, and some hormones detectable in the blood were not measurable in saliva.

Ultrasound technology has been used to extract transudate across the skin barrier. Much of this research has been directed to the detection of glucose in transudate. However, ultrasound technology has suffered from the disadvantage that the amounts of transudate extracted tend to be relatively low when compared to invasive techniques. Due to the high sensitivities associated with detection of glucose, this is not generally problematic in some applications. However, if other analytes are to be detected, detection levels can become problematic. Even in the detection of analytes with high sensitivity detectors, such as with glucose detection, the presence of excess measured glucose from previous measurements can be problematic depending on the desired accuracy of measurements to be taken on a continuous basis.

A number of additional techniques have been proposed for increasing the amount of transudate extracted. For example, U.S. Pat. No. 5,617,851 discloses a focussing means for focussing ultrasonic energy to maximise the test site. The use of variable frequency ultrasonic pumping pulses in order to cause optimal tissue permeability is also taught. U.S. Pat. No. 5,895,362 likewise discloses the use of an ultrasonic source in order to increase skin barrier permeability. In addition, it discloses the use of a partial vacuum to increase the amount of transudate extracted. U.S. Pat. No. 5,722,397 discloses the use of ultrasound together with a chemical enhancer to increase extraction volumes. U.S. Pat. No. 6,041,253 teaches that combining an electric field with ultrasound (electrosonophoresis) may further improve the movement of analytes from blood to skin surface, particularly those that are hydrophobic or complex in carrier charge. All of the above mentioned patents are incorporated herein by reference.

The above methods are often time consuming in sample methodology. None of these devices are particularly useful for continuous sampling of transudate in that they are batch-wise operated. The devices in U.S. Pat. No. 5,458,140 and U.S. Pat. No. 5,722,397 suffer from several disadvantages. The devices incorporate reservoirs of liquid chemical enhancer. These reservoirs have to be relatively large in order to dilute any transudate that passes over the skin barrier to ensure that a concentration gradient across the skin surface is maintained. In addition, if the transudate is diluted, transudate levels can be problematic to detect, or to determine accurately due to sensitivity limitations of some types of sensors. Furthermore, as transudate is not eliminated from the reservoir, changes can be increasingly difficult to monitor given that previous readings have to be subtracted from the current reading. Accordingly, the devices disclosed in these patents tend to be limited in their application for continuous monitoring purposes. Other prior art devices suffer from similar disadvantages.

WO 01/70330 provides a system for periodic or continuous monitoring of transudate analyte once the skin is made permeable by an initial treatment of ultrasound. In many respects, this device is similar to that disclosed in U.S. Pat. No. 5,722,397. The device disclosed makes use of a potential difference grid to manipulate an osmotic gradient, drawing transudate across the skin surface towards a detector. It permits the modification of an analyte of interest, thereby removing the analyte of interest from the transudate sample. However, this device does not eliminate residual transudate from the system once detection has taken place. An undesirable build-up of transudate may result.

Other difficulties of devices of the prior art are slow response times from extraction to measurement of analytes within the extracted transudate. Many devices of the prior art also fail to extract sufficient transudate for an adequate analysis to be conducted on any particular sample.

The designs of transudate ultrasonic extraction devices in the art also tend to make them difficult to adapt for multiple user sampling. One particular problem which expresses itself in a multiple user sampling environment is the need to remove a previous user's sample from the machine prior to the use by another party, or repeated use at a different time by the same party. Obviously, a main reason for removing earlier samples is to ensure accuracy of subsequent readings. A further reason for removing the sample is to minimise any possibility of infection by an infectious agent in a previous user's sample.

U.S. Pat. No. 5,895,362 provides an absorbent pad or material to receive transudate. The pad or material can be readily removed for analysis. This is an incomplete solution in that some transudate may still remain on other portions of the device. In addition, collecting transudate in this manner means that continuous sampling becomes increasingly difficult. Another approach, adopted by U.S. Pat. No. 5,458,140 and U.S. Pat. No. 5,722,397 is the use of a reservoir of liquid chemical enhancer. In a multi-user system, this approach would likely require each user to have such a reservoir attached to them which is inconvenient, time-consuming to set up and is wasteful as the reservoir contents must be disposed of each time.

It is therefore an object of the present invention to provide:

a non-invasive transudate extraction device which overcomes at least some of the above mentioned disadvantages, or that at least provides the public with a useful choice.

### SUMMARY OF THE INVENTION

In a first aspect, the invention may be said to broadly consist in a transudate extraction device for non-invasive extraction of transudate through a target area of a skin barrier of an organism subjected to sufficient ultrasonic energy to induce transudation therethrough, the device including at least:

a contacting means for contacting said target area to receive transudate therefrom,

a transudate collection chamber in open communication with said contacting means to receive transudate therefrom,

circulating means for circulating transudate within said collection chamber to thereby create and maintain a concentration gradient through the skin of a said test subject, said collection chamber having a discharge opening through which exudate collected therein can be discharged.

In a second aspect, the invention may be broadly said to consist in a method for non-invasively extracting transudate through a skin barrier of an organism, which includes at least the steps of:

treating a target area of a said skin barrier with a sufficient concentration of ultrasonic energy to induce transudation therethrough;

collecting said transudate thereby produced in a dynamic flow of fluid, said dynamic flow being controlled so as to provide a concentration gradient in a direction which induces continuing transudation through said skin barrier, and

recovering transudate from said dynamic flow.

The invention extends to a replaceable member adapted for attachment to the test area of a device of the invention.

### BRIEF DESCRIPTION OF THE DRAWINGS

The invention will be described below with reference to the Figures, wherein:

FIG. 1 is a partial sectional view of a non-invasive transudate extraction device.

FIG. 2 is a partial section through a second ultrasound collection head in association with an ultrasound source.

FIG. 3 is a graph depicting changes in concentration of testosterone as measured in serum, saliva, and transdermal samples obtained from sheep before, during, and after exercise. Means and Standard Error values for serum are in ng/ml. Means and Standard Error values for transdermal and saliva readings are in pg/ml. Saliva values represent those obtained at or up to 20 mins after both serum and transdermal samples both for pooled values (pre and post) and exercise time points.

FIG. 4 is a graph representing changes in concentration of testosterone as measured in serum, saliva and transdermal samples and cortisol as measured in serum samples obtained from human subjects before, during and after exercise who showed consistent increases in testosterone (increasers) during the exercise sessions. Means and Standard Error values for serum are in ng/ml. Means and Standard Error values for transdermal and saliva readings are in pg/ml. Saliva values represent those obtained at or up to 30 mins after both serum and transdermal samples both for pooled values (pre and post) and exercise time points.

FIG. 5 is a graph representing changes in concentration of testosterone as measured in serum and transdermal samples and cortisol as measured in serum samples obtained from human subjects before, during, and after exercise, who showed consistent decreases in the level of testosterone (decreasers) during the exercise sessions.

FIG. 6 is a partial section through a third ultrasound collection device.

FIG. 7 is a partial section through a fourth ultrasound collection device.

FIG. 8 is a partial section through a fifth ultrasound collection device.

FIG. 9 is a section through a collecting system for transudate.

### DETAILED DESCRIPTION OF THE INVENTION

The present invention provides a transudate extraction device for non-invasive extraction of transudate through a skin barrier of an organism. The device in its broadest sense comprises an ultrasonic generator, a transudate collector and a fluid circulating means.

Preferably, the circulating means comprises an inlet passage and an outlet passage in fluid communication with the chamber for flowing fluid through the inlet passage, chamber and out the outlet passage. The outlet passage is preferably connected to a detection means.

In a preferred embodiment, the body of the device is adapted to receive an ultrasound headpiece. In use, the headpiece is positioned so that ultrasound is directed to the skin of an animal when the device is placed on the animal with the contacting means applied to the skin of the animal.

In one embodiment, the method is a non-invasive continuous method for collecting transudate from an organism.

Preferably, the method and device referenced are adapted for sequential multi-organism use.

The ultrasonic generator is disposed to produce and direct ultrasonic energy to a test site on the skin barrier. The ultrasound produced by the ultrasonic generator is preferably frequency modulated from low to high, such that it generates a local pressure gradient directed out of the body. Suitable ultrasound frequencies and ranges are known in the art. In one embodiment, a preferred ranges is 1-3 MHz, more preferably about 1 MHz. In an alternative embodiment, a preferred range is 10-30 kHz, more preferably about 10 kHz. It has been found that both ranges are successful while frequencies between the two are less so.

In one embodiment, it is also preferred to have an output of 0.1 to 3 W/cm². In an alternative embodiment, an output range of 5-15 W/cm² is used. It has been found that the upper range (5-15 W/cm²) is somewhat more successful in hormonal and larger analyte collection. The lower range (0.1 to 3 W/cm²) is useful where application of ultrasound is to be minimised.

It has been found that a pulsed delivery can be as efficacious as continuous delivery within these ranges and again has benefits of reducing total ultrasound exposure. In a preferred embodiment, the pulsing regime is 5 sec on and 5 sec off. The generator may also be adapted to produce an ultrasonic standing wave across the skin barrier. The generator may also comprise an ultrasonic energy focussing means, preferably an ultrasonic lens or parabolic reflector. It is also contemplated that the generator may comprise a plurality of ultrasonic generating devices, which act in concert to produce the required ultrasonic energy. The ultrasonic generator is preferably an ultrasonic transducer. Examples of ultrasonic generators and transducers suitable for use in the present invention are known in the art. For example, those disclosed in U.S. Pat. No. 5,895,362, U.S. Pat. No. 5,617,851 and U.S. Pat. No. 5,722,397 incorporated here by reference.

The transudate collector receives transudate passing out of the organism through the skin barrier. The transudate collector may in one embodiment simply comprise a collection site where transudate is accumulated in a specific medium such as an absorbent gel. Alternatively, the collector may be a chamber or vessel. In addition, the collection site, chamber or vessel may be a nexus for a fluid stream and the transudate.

The transudate collector or skin contacting portion of the transudate analysis device to may optionally additionally comprise a permeable or semi-permeable membrane. The membrane may act as a filter preventing various substances in the transudate passing therethrough. The membrane may also act as a fluid retaining barrier to inhibit fluid in a fluid stream from leaving the transudate extraction device through the transudate collector. This may be sealed down to make the chamber watertight using a suitable sealing member, such as a rubber O ring. This allows sampling only of those components that pass through the membrane. This can avoid interference with the assay by unwanted molecules that cannot pass through the membrane (for example proteinases).

In an alterative embodiment, no membrane is provided. In such instances, a sealing member, such as a rubber O ring, may be provided on the outer surface of the chamber, which provides the chamber with a watertight seal when the chamber is placed against the skin surface of a subject.

In a preferred embodiment, the membrane is a dialysis membrane. Any suitable dialysis membranes known in the art may be employed. A preferred membrane for use is one available from Spectra Por Membranes, Houston, Tex.

The fluid circulating means generates a fluid stream, which is in fluid communication with the transudate collector to convey transudate from the test site to the collector for analysis or processing. In addition, the fluid circulating means maintains a concentration gradient across the skin barrier favourable for transudate extraction across the skin barrier. In a preferred embodiment, turbulence and dead zones in the chamber proximal the test site is minimised. Without wishing to be bound by theory, it is believed that sharp angles and obstacles offer more surface for increasing both turbulence and “dead zones” (catch spots) adjacent the membrane. More turbulence and dead zones are expected in sharp angle cases. It has been found that the best results are obtained where turbulence and dead zones are minimised. In a preferred embodiment, a circular profile, giving an overall cylindrical shape collecting chamber, is employed to this end.

In a preferred embodiment, flow rate should be in the range between 50 and 600 μl/min, more preferably 150 and 500 μl/min and most preferably about 300 μl/min.

The use of a concentration gradient formed by the fluid stream facilities transudate extraction. The presence of the gradient may obviate the need for vacuums, chemical enhancers, wave modulation and other ancillary techniques in the art to extract transudate. However, these may still be provided in order to increase transudate extraction over and above that obtained using the concentration gradient and ultrasonic treatment.

The concentration gradient across the skin barrier may be maintained or enhanced by ensuring that the fluid stream does not contain any transudate. This may be achieved in one embodiment by filtering out components of the transudate and recycling the fluid of the fluid stream or by using a continual fresh fluid stream as noted above.

The fluid circulating means may comprise a fluid inlet conduit and a fluid outlet conduit. These conduits may be in the form of tubes. The conduits at one end preferably terminate in the transudate collector. In one embodiment, these conduits terminate in the transudate collector vessel or chamber.

A fluid stream driving device is generally provided. Any suitable device in the art for causing bulk movement of fluid across the test site may be employed. Preferably, the driving device is in the form of a pump.

Control of pressure in the chamber has been found to permit optimum flow rates of transudate extraction to be obtained. It has been found that optimum conditions occur when the pressure is balanced by appropriate back-pressure from the extractions pipes through the use of a valve or shunt obstruction. Pressure in the chamber may be decreased by opening the valve in the extraction vent. Conversely, pressure in the chamber may be increased by partially closing the valve.

In an alternative embodiment, instead of valve of shunt obstructions, an electronic control system which balances an inlet pump and an outlet pump is employed. The outlet pump may also be replaced with a dynamically alterable exhaust valve.

The fluid stream may comprise a continual stream of fresh fluid. Alternatively, fluid in the fluid stream may be stored or recycled as noted above. Particularly in this latter case, the fluid in the fluid stream may be filtered to remove previous transudate samples or components of the samples. A variety of filtering means are available in the art and may be used. It is also contemplated that the fluid stream may be separable from transudate because of differing physical properties, such as phase.

The fluid in the fluid stream may comprise a gas or a liquid. Again, a variety of carrier gases or liquids known in the art may be used. Examples of preferred liquids include water, saline, diols, such as propylene glycol and glycerol; mono-alcohols such as ethanol propanol, and higher alcohols; DMSO; dimethylformamide; N,N-dimethylacetamide; 2-pyrrolidone; N-(2-hydroxyethyl) pyrrolidone, N-methylpyrrolidone, 1-dodecylazacycloheptan-2-one and other n-substituted-alkyl-azacycloalkyl-2-ones (azones). Gases such as air and nitrogen are also contemplated. Preferably a pharmacologically acceptable carrier liquid, such as saline, is used. In one embodiment, 10% ethanol is employed.

In one embodiment, the present invention also provides cleaning means for removing excess transudate from the transudate extraction device. This cleaning means may be combined with the fluid circulating means. This cleaning means also reduces the risk of between subject contamination. In this embodiment, it is preferred for the fluid stream to be germicidal or antimicrobial. Examples include 3^{rd} generation germicides such as Hibitane™ (chlorhexidine) and 70% ethanol. The fluid circulating means may also be designed in such a way as to cause an excess of fluid to build up at the transudate collector in order to wash the site of any residual transudate after use.

Alternatively, the head may be replaced by other similar or identical heads for each subject to be tested. The used heads may be discarded or reused after sterilisation and cleaning. This may be used to ensure that head is sterile and that no residual transudate from a previous subject is present which may give rise to erroneous readings.

The transudate extraction device may also be provided with a detector or analyser for detecting the presence of, and/or measuring the level of one or more substances of interest in a transudate sample. Polar and charged moieties are preferred. Non-limiting examples of substances which may be detected and analysed using the extraction device of the invention include proteins, polypeptides, steroid hormones, carbohydrate moieties, and metabolites but are not limited thereto. More specific non-limiting examples are selected from the group comprising caffeine, ethanol, progesterone, human chorionic gonadotropin, prolactin, procalcitonin, TNF alpha, IL 6, propofol, pseudoephedrine, insulin, interferon, oestrogen, testosterone, 17-β estradiol, cortisol, corticosterone in rodents, erythropoetin, glucose, ethanol, caffeine and lactic acid. Although it is likely there will be some size and charge limitations on analyte movement and recovery other specific, but non limiting examples, include immunoglobulins, beta-2-agonists, beta blockers, androstenediones, decadurabolin, dehydroepiandrosterones, stanozolol, diuretics, adrenocorticotrophins, amine based substances (eg. catecholamines), amino acids and amino acid based neurotransmitter substances, choline based substances, creatine by-products, corticotrophin releasing factors, insulin, insulin like growth factors, somatomedins, leutinising hormone, amphetamines, cannabinoids, opioids, endorphins and enkephalins, thyroid hormones, antibiotics, both local and general non volatile anaesthetic agents, some markers of both bacterial and viral infection including infective agent itself, cytokines, chaperone proteins particularly heat shock proteins. Amino acids, dextrose, fructose, sucrose, ionic salts, free fatty acids, lactates, creatine phosphate and kinases are also likely, but non-limiting examples of analytes collectable and measureable.

The transudate extraction device may further comprise a vacuum chamber, a chemical reservoir and/or a wave modulation means all as set forth in the cited specifications, in the background of the invention and as noted above.

In a preferred embodiment the device, alternatively portions thereof, are housed in a moulded plastics body which may define various functional elements of the invention.

Processing, detection and/or storage devices are also contemplated. Example of detectors include spectrophotometric, infra red, temperature, magnetic resonance, atomic absorption, mass spectroscopy, pH, electrochemical and conductivity detectors. Real time sensors for real-time analyte monitoring are particularly preferred. Examples include immunosensors.

Preferably the relative volume of transudate and fluid mixture brought into contact with the surface area of the detection devices exposed to fluid is large, more preferably a long length of sensor detection relative to the flow containing the transudate. For example the sensor may be 5 mm long and in contact with 500 μl or less fluid via a thin flow pass tube.

Examples of storage devices include sample phials, and volumetric or time-based sample collectors. Examples of processing devices contemplated include cooling (preferably freezing), heating, preserving and chemical or enzymatic treatment devices and separating means like columns, filters, HPLC and electrophoretic gels such as are known in the art. Processing devices and detectors may be combined, such as in enzyme-linked assays. Combinations of these devices are also contemplated. In a preferred embodiment, the processing devices are mass spectroscopy and electrochemical detectors and the processing device is a HPLC separating means.

Suitable subjects that the invention may be used with vary widely. Testing of transudate from agronomically important animals such as sheep, cattle, deer, goats, pigs and fowls as well as pets (including dogs and cats and birds) are contemplated. However, testing of transudate from humans is the preferred application. Other non-limiting suitable organisms may be selected from: rats, horses and sharks. Another key advantage is in wildlife and conservation studies where non-invasive sampling lowers the risk to the animal. A key but non-limiting example is endangered birds. There is an advantage in reduced stress of sampling on the organism.

Test sites anywhere on the body of the subject being tested are feasible and the size may be varied across a broad range. Usually a site on a limb of between 1 and 20 cm² will be used, preferably the site is 5 cm² or less.

Generally, the devices of the invention will be set up for static use in a location such as a doctors room, gym or for home use. However, it is contemplated that the device in its entirety and/or portions of the device may be affixed to a user for wearable or portable use. In one embodiment, the transudate collector and fluid circulating means may be carried by a user or affixed to a user. Accordingly, kits comprising portions of the device are also contemplated.

In use a small amount of ultrasound gel is rubbed into the skin area of collection. This may be done 10 minutes prior to the extraction. An initial application of ultrasound may be given to start the transdermal flux. Typically the time of initial application is 0.5-2 minutes.

In a further aspect, the present invention provides a method of non-invasively extracting transudate from an organism through a skin barrier of the organism. The method comprises treating the skin barrier with ultrasonic energy sufficient to permit transudate to move through the skin barrier. In addition, the method comprises removing the transudate through the skin barrier surface by conveying it in a fluid stream to maintain a concentration gradient favourable to further extraction of transudate across the skin barrier.

In a continuous flow system, the process of treating the skin with ultrasonic energy and removing the transudate may be performed simultaneously. Alternatively, these processes may be independent from one another. The ultrasonic energy may be produced using an ultrasonic generator as hereinbefore described. However, the present invention is not limited to the specific embodiments disclosed herein and any form of ultrasonic energy sufficient to permit the transudate to move through the skin barrier may be utilised. The fluid stream may also be as defined above.

Other methods and devices in the art may be employed to increase the extraction rates and volumes of transudate across the skin barrier. Examples of these include wave modulation, partial vacuum systems and chemical enhancers such as are taught in U.S. Pat. No. 5,895,362 and U.S. Pat. No. 5,722,397, which are incorporated herein by reference. In a preferred embodiment, the present invention makes use of an electrosonophoresis system as taught in U.S. Pat. No. 6,041,253, which is incorporated herein in its entirety by reference. This system employs an electromagnetic field to encourage analyte extraction across a biological membrane. The Voltage across the electrodes may conveniently be selected from the range 3-15 volts. In a preferred embodiment, 9V is employed.

Therefore, the body of the device may additionally include a skin-permeability-increaser. Any suitable device available in the art to increase skin permeability to permit an increased flow of transudate is contemplated. Suitable means include, but are not limited to, ultrasonic generators, electroporation means, iontophoresis means, chemical agents and lasers. However, the skin may also be pre-prepared to encourage transudate passage prior to use of the device of the present invention, thereby obviating the need for a dedicated skin-permeating means to be present in the device of the present invention.

The devices and methods of the invention have a broad range of applications. Examples include, but are not limited to: patient monitoring; drug and substance abuse detection; detection of therapeutic agents, antibiotics, anaesthetics and residues, toxic substances; detection of menstrual cycle or oestrous via hormone levels; detection of stress levels via cortisol; and detection of other human performance and drug states. The devices and methods are particularly useful in sports performance monitoring and worker stress monitoring. Use of the devices and methods enable quick, continuous, and painless extraction of transudate.

Between subjects and cleanup sterilisation occurs of both the head, collecting tubes and the membrane

Membranes may also be changed between subjects

Membranes may also be changed both between and within subjects to increase or decrease membrane molecular weight cutoff points that then can dictate analytes crossing the membrane into the collection chamber. This can act to selectively cut down on artefact collected when analytes of particular molecular size are of interest.

The entire head piece can also be interchanged between subjects as it sits atop the ultrasound delivery device, each head piece locking firmly onto but disconnectable from the ultrasound device.

Alternatively the head may also contain chambers of different sizes to allow the collection volume exposed to the skin analytes to be altered. This can alter osmotic flow properties and concentration of analyte in the collection fluid. Alternatively or in combination with this varying perfusion flow through the chamber can alter these properties.

The invention is further described below with reference to the accompanying figures and the examples. This disclosed embodiment is not intended to be limiting to the invention.

### DETAILED DESCRIPTION OF THE DRAWINGS

### Construction and Operation of a Device According to a First Aspect of the Invention

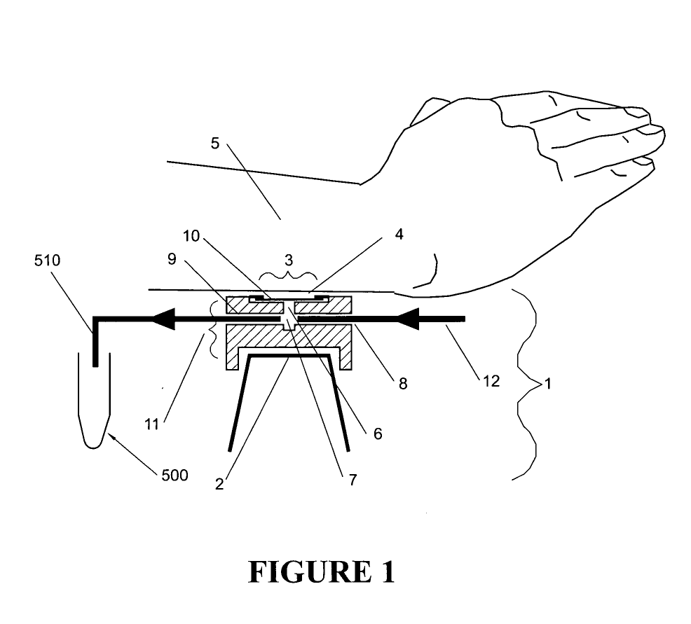

With reference to FIG. 1, a transudate extraction device (1) comprises a US-3 Model ultrasonic generator (2) from Ito Co., Ltd, Tokyo, Japan for generating ultrasonic energy at a test site (3) at the skin barrier (4) on the forearm of a person (5). A transudate collection chamber (6) leads into a fluid stream/transudate nexus (7). A fluid stream (12) is provided by means of a fluid stream inlet (8) terminating at the transudate/fluid stream nexus (7). A fluid stream outlet (9) is provided extending from the nexus (7). A semi-permeable dialysis membrane (10) from Spectra Por Membranes, Houston, Tex. separates the test site (3) from the chamber (6). A body (11) retains all the components of the device (1) in relationship to one another.

In use, a person places their forearm (5) onto the device in contact with (1). A fluid stream (12) is introduced into the fluid stream inlet (8), and the ultrasound generator (2) is activated. The fluid stream (12) comprises physiological saline in the preferred embodiment. Other suitable examples include solutions of ethanol or glucose.

The ultrasonic energy focuses on the test site (3) causing the skin barrier (4) to become permeable. Transudate(not shown) moves across the skin barrier (4) from the forearm (5) of a person down a concentration gradient set up over the skin barrier (4) and dialysis membrane (10) into the chamber (6). The transudate merges with the fluid stream at the nexus (7) with the fluid stream (12) and is drawn out of the device (1) via the outlet (9).

The transudate enriched fluid stream is now analysed, processed or stored according to the requirements of the system. It will be appreciated that due to the fresh introduction of fluid in the fluid stream and the removal of transudate, the concentration gradient will be maintained even after transudate has been extracted.

Transudate coming through the outlet (9) is stored in a collection vial (500). In more detail, vial 500 has a sealing flange (502) and cap (503). Tube (510) is attached one end to outlet (9) on one end and on the other end passes through an adapter, generally indicated as (505) and terminates at (512). Adapter (505) has a body (507) with a sealing flange (509), outlet tube (510), and pressure equalisation tube (515).

In use, sealing flange (509) is sealed against the complementary sealing flange (502) on vial (500). Transudate coming from outlet (9) passes through outlet tube (510) and drops into vial (500) by operation of gravity. At the end of collection, the vial is disconnected from adapter (505) and cap (503) is closed over sealing flange (502). The tube is then stored as appropriate to the analyte of interest at room temperature, 4° C. and −20° C. When an analysis machine becomes available, the samples in the tubes are analysed by standard means, including HPLC, Mass spectrometry, ELISA and RIA.

Once the desired transudate has been extracted, the person may remove their forearm from the device. The ultrasonic generator and fluid stream may then be deactivated.

In order to reset the machine for the next person, the fluid stream may be reactivated to flush the chamber (6), and nexus (7) of any residual transudate. In order to flush the transudate collection site, it may be necessary to restrict the outlet (9) to force fluid of the fluid stream (12) through the dialysis membrane (10). Another method of flushing the device is to increase the fluid flow rate to build up pressure in the nexus (7) and collection chamber (6) thereby forcing fluid from the fluid stream over the dialysis membrane (10). Alternatively, the device may be cleaned by conventional means such as by wiping.

### Construction and Operation of a Second Embodiment of the Invention

A collection head (generally indicated by 50) is made by hollowing out a solid block of acetal polyformaldehyde plastic (55) towards one end (generally indicated by 60) where to leave a 0.5 cm thick peripheral wall (65). The hollowed out end (60) is designed to fit tightly over an adapted commercial hand-held ultrasound device (70). The device (70) is an ITO Physiotherapy and rehabilitation ultrasound unit, Tokyo 176-8605, Japan.

At the solid end of head (50), a small chamber (75) of 300 μl volume is constructed. The end face (80) is further recessed by another 0.2 cm around chamber (75). The chamber has an entry (85) and exit port (90) fed by polyethylene tubing (95 and 100) allowing for a constant flow of perfusion. An adapted semi-permeable dialysis membrane (105) by Spectra/Por, Spectrum Lab., CA 90220-6435, USA, is sealed by a rubber O ring (110) making the chamber watertight. This end (80) of the head (50) is for contacting with skin (not shown) during sampling.

On either side of the head (50), button electrodes (112 and 115) are provided. These electrodes are connected to a 9V battery or other 9V supply for supplying a 9V driven electric field. Ultrasound conducting gel (120) is provided in the interface between the chamber (75) and the ultrasound device (70). The ultrasound device (70) is fitted tightly into the hollowed out end (60) as far as it will penetrate towards the chamber (75).

Four immunosensors (125) are positioned in tubing (100) in series proximal the exit (90) of the sampling head chamber (75). The immunosensors (125) have been used in vivo to measure, in real time, hormonal changes in brain tissue and circulation of conscious behaving animals. These immunosensors are enclosed within a dialysate membrane, which affords a preliminary sample separation, and use osmotic equilibration principles to maximise sample collection within a very small volume of fluid.

Within each immunosensor (125) itself, when an antibody detection of analyte is used, there is a large antibody surface area available relative to the volume of circulating analyte, with the antibody binding and detection performed on the measuring electrode. This arrangement facilitates capture and measurement at low concentrations (femtograms/ml [fg/ml]).

This consequently allows collection over very short sample times. The immunosensor itself is very small allowing it to be positioned in a relatively non-obtrusive fashion. Combining such technology with non-invasive sampling permits on-line monitoring, non-invasive, device to be constructed.

The immunosensors are manufactured in accordance with the teachings in Cook C J., Real-time measurements of corticosteroids in conscious animals using an antibody-based electrode. Nat. Biotechnol 1997;15:467-72; Cook C J., Real-time measurements of neurotransmitters in conscious sheep J. Neurosci Methods 19967;72:161-6; and Cook C J., Monitoring on-line of extracellular gamma-amino-4-butyric acid using microdialysis coupled to immunosensor analysis. J Neurosci Methods 1998;82:145-50. The disclosures of these publications in their entirety are incorporated herein by reference.

Antibodies to the analyte of interest (for example cortisol) are fixed within a dialysate probe, or in the effluent of the probe. The detection system is based upon a competitive reaction of endogenous and peroxide (HRP) labelled exogenous analyte with specific, to the analyte, antiserum immobilized on the platinum electrode housed within the dialysis membrane. After binding, a peroxidase reaction is initiated and substrate oxidation is monitored by the change in current between the electrodes in the probe, which have been set to a voltage sufficient to ensure oxidation. The bound HRP is activated by aminosalicylic acid, and the resultant electrical output of this activation is monitored. This output is proportional to the bound HRP and is therefore inversely proportional to the bound analyte of interest.

The platinum electrode acts simultaneously as immunoreagent and electrochemical detector, a combination producing increased assay sensitivity. Surrounding the electrodes with a dialysis membrane, adapted for use, allows continuous measurement in vivo without the need for sample withdrawal. Active constituents diffuse across the dialysis membrane in the direction of osmotic equilibration drive.

Briefly, each immunosensor consists of an adapted outer dialysis membrane (100 K MW cut-off), 4 mm in length, used to enclose a central platinum electrode (working electrode) and two further electrodes situated further up the probe, a reference (Ag/AgCl) and counter (Ag) electrode. Polyclonal antibodies for cortisol, testosterone, 17-β estradiol and insulin (Sigma) were absorbed onto the surface of the platinum electrode by cathodic copolymerisation, as previously described, of four different probes, respectively. In vitro and pilot in vivo studies (data not shown) suggests antibody dilutions of 1:20 cortisol, 1:20 testosterone, 1:10 17-β estradiol and 1:10 insulin for human subjects, and 1:20, 1:15, 1:5, 1:10, respectively, for sheep give appropriate sensitivities over the range of measurement.

The electrode configuration is set at 750 mV versus the Ag/AgCl reference and probes are perfused at 2 μl/min 10% ethanol using a syringe pump. For humans and sheep, calibration should be performed over the ranges of 0.1-100 ng/ml cortisol, for testosterone 25-1000 pg/ml, for 17-β estradiol 0.1-20 pg/ml and for insulin 0.1-5 ng/ml for sheep and 0.1-25 μU/ml for humans. Immunosensors are also calibrated for interference, cross reactivity, changes with pH and oxygen content and washout of measured analytes.

Immunosensors (125) are positioned in the exit line (100) of the collection head (50) in series, and fluid containing varying concentrations of testosterone, 17-β estradiol, insulin and cortisol pumped through line (95) into the collecting head chamber (75) and out exit line (100) to ensure probe calibration is maintained in vivo. In addition to continuous in-line measurement, batch samples may be collected by collecting fluid from the exit line (100). The in-line measurement by the immunosensors (125) may be switched off while batch samples are collected. This prevents interference of the immunosensors (125) with later off-line analysis of the batch collected samples. Conveniently, off-line measurements are used to calibrate the immunosensors (125) before and after each use.

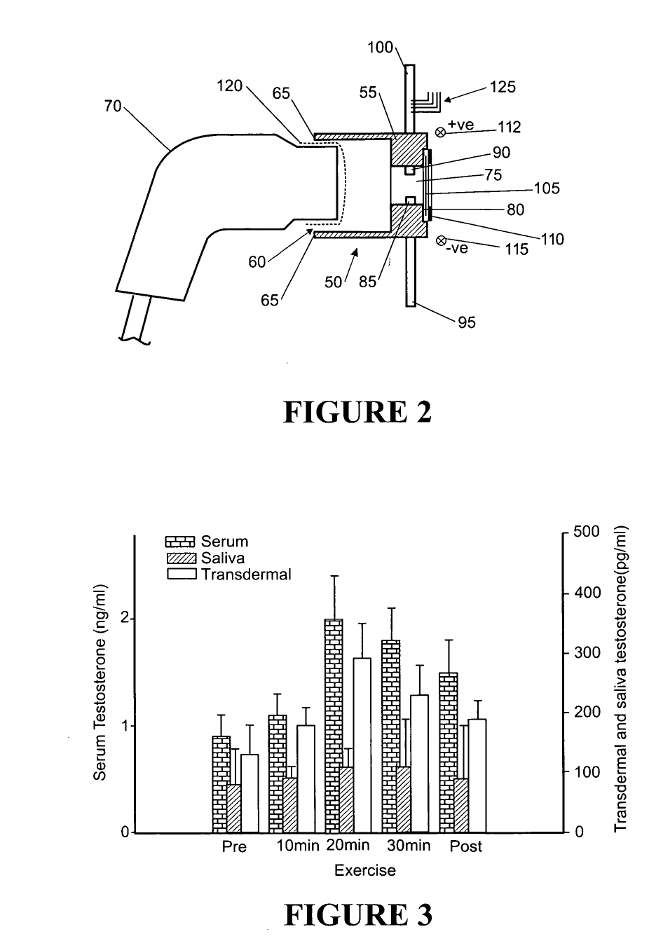

The device illustrated in FIG. 2 provided in vitro recoveries between 23% and 35% for the hormones sampled in the following examples. Washout curves gave no evidence of any of the hormones being retained on the dialysis membranes when the working electrode and electrosonophoresis had been active. There was a slight retention of hormones (between 1% and 7% of total recovery) when immunosensors/electrosonophoresis were not active. However, this retention when it occurred was constant across measurements and did not affect the linearity of response.

The immunosensors (125) showed linear responses to the ranges of hormones outlined in the examples below. In the immunosensors designed for sheep, sensitivities of 1-2 nA/ng/ml cortisol, 1-2 nA/pg/ml testosterone, 1-2 nA/10 pg/ml insulin and 1-2 nA/0.1 pg/ml 17-βestradiol were seen. Limits of detection were 0.5 ng/ml, 1 pg/ml, 0.1 pg/ml and 0.1 pg/ml, respectively. Greater than 90% of response was seen in less than 30 s (27.3±4.9, mean±S.E.M.) for the hormones as a group. A pH below 6.0 decreased sensitivity but did not affect linear responses. Changing oxygen levels had no effect, and increasing K⁺ increased the background slightly (approximately 0.2 nA for each additional 100 mmol/l K⁺). Ascorbate had no effect as the immunosensors were coated with phenylenediamine (excludes electrointerference from ascorbate).

Immunosensors designed for the human subject showed similar performance characteristics, however, sensitivities were 1-2 nA/0.1 ng/ml cortisol, 1-2 nA/10 pg/ml testosterone, 1-2 nA/0.5 μU/ml insulin and 1-2 nA/pg/ml 17-β estradiol.

Immunosensors showed stability at room temperature for >48 h or at least 800 measurements.

### Operation of the Second Embodiment

A small amount of ultrasound conducting gel is rubbed into the target skin area (not shown) of a subject 10 minutes prior to the procedure of transudate extraction. An initial application of ultrasound from the ultrasound device (70) for 1 minute is then given to the skin area (not shown) to start the transdermal flux. Applying standard ultrasound gel (120) to the end of the adapted commercial ultrasound gun (70) before positioning within the collection head (50) desirably increases ultrasound coupling.

The chamber (75) in the head is filled with 10% ethanol. The head is then positioned against the skin surface of the skin area and 1 min of ultrasound (output 20 kHz, 10 W/cm² calculated at skin surface, pulsed 5 s on/5 s off) applied. At the completion of 1 minute, fluid is allowed to flow through the chamber at a rate of 300 μl/min for a further min while the device remains in contact with the skin. During the full 2 min contact with the skin, a 9V supply is applied across terminals (112 and 115) to create an electric field across the head at the skin surface.

Immunosensor (125) measurement takes a further 1 minute. Then both the chamber (75) and immunosensors (125) are flushed taking another 1 minute. Ideally, repeat measures could be obtained every 4 min using this cycle. The semi-permeable membrane (105) is changed manually if used on separate subjects to avoid cross contamination. This adds time to the cycle.

Subjects are staggered in timing to allow measurements on a 5-min basis when necessary and to maintain a constancy of time across the experiment.

For saliva sample analysis, the head (50) is not connected to the ultrasound device (70), and saliva is fed straight into the collection chamber (75) and flowed through to the immunosensors (125).

### Description of a Third Embodiment

A sample collection head, generally indicated as (190) comprises a body (195) into which inlet (200) and outlet (205) ports are formed. The inlet (200) leads through a passage (207) to a chamber (210). The chamber (210) is connected to an outlet passage (212) to the outlet port (205). Two electrodes (215) and (220) are provided on the body (195).

Chamber (210) has an opening (222) covered by a releasable cover (225). The cover (225) is connected to the body (195) by means of a screw-threaded engagement (230). The cover (225) also has a semi-permeable membrane (235) held in position by a sealing O-ring (240). An ultrasound head abutment (245) is defined between the two ports (200) and (205).

In use, cover (225) and electrodes (215 and 220) are placed against the skin (not shown) of an organism from which transudate is to be taken. An ultrasound generator (not shown) is placed against abutment (245). Ultrasound energy is directed to the skin (not shown) proximal opening (222). A 9V potential difference is applied across electrodes (215 and 220). Physiological saline (not shown) is pumped into the inlet (200), through passage (207) into chamber (210). Transudate (not shown) from the skin of the organism passes through membrane (235) into chamber (210), where it mixes with the saline. Transudate loaded saline moves from chamber (210) into passage (212) out of outlet (205).

After use, cover (225) is unscrewed it from its screw-threaded engagement (230) with body (195). Cover (225), including membrane (235) and O-ring (240), is sanitised for re-use or discarded.

### Description of a fourth Embodiment

A sample collection head, generally indicated as (290) comprises a body (295) into which inlet (300) and outlet (305) ports are formed. The inlet (300) leads through a passage (307) to a chamber (310). Chamber (310) is connected to an outlet passage (312) to the outlet port (305). Two electrodes (315) and (320) are provided on the body (295).

Chamber (310) has an opening (322) covered by a releasable cover (325). The cover (325) is connected to body (295) by means of a resilient snap-fit engagement (230) comprising an annular ring (332) on body (295) and a complementary indentation (333) cut-out on the inner surface of cover (325). The cover (325) also has a semi-permeable membrane (335) held in position by a sealing O-ring (340). An ultrasound head abutment (345) is defined between the two ports (300) and (305).

In use, cover (325) and electrodes (315 and 320) are placed against the skin (not shown) of an organism from which transudate is to be taken. An ultrasound generator (not shown) is placed against abutment (345). Ultrasound energy is directed to the skin (not shown) proximal opening (322). A 9V potential difference is applied across electrodes (315 and 320). Physiological saline (not shown) is pumped into the inlet (300), through passage (307) into chamber (310). Transudate (not shown) from the skin of the organism passes through membrane (335) into chamber (310), where it mixes with the saline. Transudate loaded saline moves from chamber (310) into passage (312) out of outlet (305).

After use, cover (325) is snapped off body (295) at the snap-fit engagement (330). Cover (325), including membrane (335) and O-ring (340), is sanitised for re-use or discarded.

### Description of a Fifth Embodiment

A sample collection head, generally indicated as (390) comprises a body (395) into which inlet (400) and outlet (405) ports are formed. The inlet (400) leads through a passage (407) to a chamber (410). Chamber (410) is connected to an outlet passage (412) to outlet port (405). Two electrodes (415) and (420) are provided on body (495).

Chamber (410) has an annular opening (422) flanked by an O-ring (430) standing proud of a retaining recess (435) in body (395).

In use, O-ring (430) and electrodes (415 and 420) are placed against the skin (not shown) of an organism from which transudate is to be taken. Sufficient pressure is applied to the skin to ensure a seal is formed between the skin and O-ring 435. An ultrasound generator (not shown) is placed against abutment (445). Ultrasound energy is directed to the skin (not shown) proximal opening (422). A 9V potential difference is applied across electrodes (415 and 420). Physiological saline (not shown) is pumped into the inlet (400), through passage (407) into chamber (410). Transudate (not shown) from the skin of the organism passes into chamber (310), where it mixes with the saline. Transudate loaded saline moves from chamber (410) into passage (412) out of outlet (405).

After use, O-ring (430) is removed from recess (435), is sanitised for re-use or discarded.

### EXAMPLE 1

### Trial

The device of shown in FIG. 1 has been trialed with four individuals. The device was assembled as set out above and was linked to a testosterone and ethanol detectors comprising a HPLC system and Electrochemical detector and a mass spectroscopy system. The subjects were four male athletes, with body weights between 82 and 106 kilograms, aged between 21 and 24 years. In turn, each subject placed their forearm on the device. The ultrasound generator was set to produce continuous pulsing, collimating ultrasonic energy at 1 MHz and 0.5 W/cm². The surface of delivery at the test site was approximately 5 cm². Collections were made from the skin surface of the forearm over a period of one minute. Blood samples were drawn simultaneously and analysed for comparison to the ultrasound samples. The results are set out in Table 1.

TABLE 1

| | | Ultra- | | Ultra- |
|---|---|---|---|---|
| | Blood level | sound | Blood | sound level |
| | 1# | level 1# | level 2* | 2* |
| Subject 1 | Testosterone | Testosterone | Testosterone | Testosterone |
| 82 kg | 4 ng/ml | 2 ng/ml | 6 ng/ml | 3 ng/ml |
| 21 years of | Ethanol | Ethanol | Ethanol | Ethanol |
| age | 1 pg/ml | 0.7 pg/ml | 0.6 pg/ml | 0.3 pg/ml |
| Subject 2 | Testosterone | Testosterone | Testosterone | Testosterone |
| 87 kg | 3 ng/ml | 2 ng/ml | 5 ng/ml | 4 ng/ml |
| 22 years of | Ethanol | Ethanol | Ethanol | Ethanol |
| age | 0.5 pg/ml | 0.3 pg/ml | 0.8 pg/ml | 0.5 pg/ml |
| Subject 3 | Testosterone | Testosterone | Testosterone | Testosterone |
| 106 kg | 7 ng/ml | 4 ng/ml | 10 ng/ml | 6 ng/ml |
| 24 years of | Ethanol | Ethanol | Ethanol | Ethanol |
| age | 0.5 pg/ml | 0.2 pg/ml | 0.4 pg/ml | 0.2 pg/ml |
| Subject 4 | Testosterone | Testosterone | Testosterone | Testosterone |
| 95 kg | 9 ng/ml | 7 ng/ml | 8 ng/ml | 4 ng/ml |
| 23 years of | Ethanol | Ethanol | Ethanol | Ethanol |
| age | 0.5 pg/ml | 0.3 pg/ml | 0.9 pg/ml | 0.3 pg/ml |

#readings taken on day 1 30 min following 1 hour training (aerobic) and oral administration of 10% ethanol.
*repeat of above on day 3.

The above results indicate that there is some correlation between the blood levels and the ultrasound levels. The discrepancies may be ascribed to differences between the fluids in the blood stream and fluids extracted from capillaries, veins and the lymph system. Specifically, there is a definable relationship between the blood levels of these analytes and those levels detected by ultrasound.

### EXAMPLE 2

### Analysis of Blood and SLS-UG and SLS-EG Samples

Blood samples were taken from subjects by well-established methods. Some examples of these techniques are more fully set out in the examples below. The samples are centrifuged, separated and stored at −20° C. until assay, using techniques well known in the art.

Insulin, 17-β estradiol, testosterone and cortisol concentrations, both free and total in serum, were assayed by ELISA or RIA (Elisa Kits, DRG Instruments, D-35039 Marburg, Germany, RIA constituents, Sigma, St. Louis, Mo. 63178, USA) and glucose by a spectrophotometer kit (Sigma).

Sodium lauryl sulfate (40%) and either a commercial ultrasound conductive gel (Aquasonic, Parker Lab., NJ 07004, USA) or a 10% ethanol gel (the two gels were alternated in use) are employed. These gels are designated SLS-UG and SLS-EG respectively herein. Collected SLS-UG and SLS-EG are assayed as for blood. Saliva was divided into two samples. The first sample is stored at −20° C. and assayed as for blood. The second sample is assayed at the time of experimentation using the apparatus and immunosensors described above.

### EXAMPLE 3

### Statistical Analysis

Student t tests, ANOVA or Wilcoxon test for paired samples are made as appropriate. Statistical significance is taken at P≦0.05 level. For analysis across the exercise stress, data is grouped into pre, during and post exercise values as actual sample times relative to the exercise for transdermal and blood obtained values. For pre, during and post exercise grouping of saliva samples, a 20-min lag was allowed relative to blood and transdermal values in sheep and a 30-min lag in human subjects.

### EXAMPLE 4

### Romney Sheep

The purpose of this example was to combine two techniques of non-invasive sampling: transdermal exudate facilitated by electrosonophoresis and saliva collection by bulb suction, with rapid measurement using appropriate immunosensors and comparing their use. In the case of the electrosonophoresis, the sampling and measurement components were constructed as one hand-held device. For saliva, the collected volume was added to the measurement part of this device. Testosterone, cortisol, estradiol and insulin were chosen as the analytes because of their broad endocrinological roles and interest. The hormones were followed across an exercise stress as this changes their levels in circulation in a short period. Glucose was chosen as a hydrophilic marker, again of broad relevance.

Twenty sheep (Romney cross ewes, 36-44 kg live weights) were farmed together as a flock for 14 days prior to experimentation. On each of these days, they were rounded up, penned, a patch on their backs (approximately 5 cm in diameter) shaved and SLS-UG or SLS-EG applied.

Two minutes later the electrosonophoresis device was placed briefly on this patch (not turned on). At the end of 1 h of penning saliva was collected using a plastic suction bulb positioned manually into the mouth of the sheep. In this manner the sheep were familiarized to the experimental procedure. Collected saliva was analysed for cortisol concentration as previously described.

Ten of these animals were used to develop the transdermal exudate collection technique. These 10 animals were penned as above, and a catheter inserted into the jugular vein using known techniques in the art. A mixture of sodium lauryl sulfate and either SLS-UG or SLS-EG was positioned onto the shaved area on the back of each animal. The gel remained on this area for 1 h; replenished occasionally (the ethanol containing mix tended to dry out over a 15-20-min time span), during which time three blood samples were also taken. At the end of the hour, the remaining mix on the back was collected. The entire procedure was repeated three times. This mix was subject to assay (see below) for hormones and glucose content.

On the following day, one of the two gel mixes were rubbed on and, after 10 min, ultrasound (20 kHz, 10 W/cm², pulsed 5 s on/5 s off using an ITO Physiotherapy and rehabilitation unit, Tokyo 176-8605, Japan) applied for 1 min directly onto the mix. Ten minutes later, the mix was again collected and measured. This was then repeated with 2 min of the ultrasound application, and on a following day, the entire procedure repeated using the alternative gel mix on the shaved patch on the animal. Times greater than 2 min were not attempted due to potential risk of skin damage. Blood samples were collected at the time of ultrasound application and again at the time when the gel mix was also collected. The entire ultrasound procedure was then repeated on subsequent days with the addition of an application of a 9V electric field either side of the ultrasound head (in contact with skin).

On a separate day, in a further repeat of the above procedure; the collecting head (50) was positioned onto the ultrasound device. The ultrasound device was modified to ensure good coupling for use with the collecting head and delivery of needed parameters to the skin. The output of the device was reset to achieve the same energy and pulse delivery to the skin's surface as when the collecting head was not present, and the ultrasound device had been placed directly in contact with the gels (as described above). Three fluid flow rates into the collection chamber of the head were compared: 150, 300 and 600 μl/min. The effluent fluid was then assayed. On a final day, this was then repeated with the immunosensors positioned in the exit line from the chamber, with immunosensor measurement of the hormones made as has been described above.

From these experiments, parameters for transdermal collection were set. These were used in the both the human and sheep subjects undergoing the exercise stress.

In the remaining 10 sheep, experimentation took place on three separate days, each 3 days apart. On days of experimentation, animals were penned as above and the jugular vein of the animal was catheterised. Animals were left to rest for 50 min and then a single ultrasound application applied for 1 min as above. This initial ultrasound application was necessary to start the transdermal flux (data not shown). Provided the application was given 10 min prior to the start of further experimentation, it allowed subsequent transdermal collections to be made concurrently with blood sampling (i.e. no time lag between the two samples). A series of electrosonophoresis collections were made concurrently with blood collection from the jugular vein, every 5 min for a further 1 h. Saliva samples were collected on every second blood sample (i.e. every 10 min). At completion of this time, a human shepherd, intermittently for 30 min, then ran the animals around a paddock. During this 30 min, each animal was briefly restrained manually and sampled on all three parameters every 10 min (i.e. three samples repeated three times). At the end of this period, animals were repenned and rested for 10 min, and a further saliva collection was made. Following this saliva collection for a further 1-h period, collection as above, prior to exercise, was repeated. In this manner for each animal, in each session, 27 blood and transdermal samples and 16 saliva samples were collected.

In the mix of SLS-UG, or SLS-EG, alone (without electrosonophoresis), only glucose was measurable between 0.05% and 0.09% blood serum levels. The addition of ultrasound application increased the glucose recovery considerably (to approximately 4%), and both cortisol and testosterone were reliably detectable at 5% and 2% serum levels. Insulin and 17-β estradiol were detected (2% and 1%, respectively, of serum levels) but were not always reproducible at this level in each animal. The three hormones, when detected, were in higher concentrations in the SLS-EG mix than in the SLS-UG mix, although the mix had no effect upon detectable glucose. In each case, a short burst of ultrasound (1 min) was needed to start the transdermal flux, and a delay of 10 min before full concentration was achieved. Once the flux was established, there was no lag in time between blood and transdermal samples within the measurement cycle of 4 min, however, application of ultrasound was needed at least every 30 min (data not shown) to maintain this. In practice, ultrasound was applied on each measurement.

The addition of an electric field to the ultrasound improved recovery of glucose concentration slightly (7%) and greatly increased the concentration measured of all four hormones (Table 2a and 2b), allowing reproducible detection. This was further enhanced by the use of the collecting head containing the adapted membrane (Table 2a and 2b).

TABLE 2a
Concentrations of hormones and glucose in transdermal
exudate collected by different formats from sheep

| | Glucose | | Testosterone | | Estradiol | |
|---|---|---|---|---|---|---|
| | Serum, | Exudate, | Serum, | | Serum, | Exudate, |
| | mg/l | mg/l | ng/ml | Exudate, ng/ml | pg/ml | pg/ml |
| SLS + UG | 844.6 ± 81.2 | 0.61 ± 0.11 | 1.1 ± 0.6 | ND | 2.3 ± 1.9 | ND |
| | | (0.07%) | | | | |
| SLS + 10% E | 751.4 ± 110.9 | 0.59 ± 0.87 | 0.9 ± 0.4 | ND | 1.4 ± 1.6 | ND |
| | | (0.08%) | | | | |
| SLS + 10% E + US | 910.8 ± 120.4 | 36.4 ± 5.6 | 0.9 ± 0.3 | 0.02 ± 0.005 | 1.9 ± 1.1 | 0.05 ± 0.04 |
| 1 min | | (4%) | | (2%) | | (2%) |
| SLS + 10% E + US | 808.5 ± 80.9 | 59.7 ± 10.4 | 0.7 ± 0.6 | 0.05 ± 0.02 | 2.0 ± 0.8 | 0.09 ± 0.05 |
| 1 min + 9 V | | (7%) | | (7%) | | (4%) |
| SLS + 10% E + US | 890.3 ± 99.7 | 80.1 ± 11.5 | 0.8 ± 0.6 | 0.11 ± 0.06 | 1.3 ± 1.0 | 0.11 ± 0.05 |
| 1 min + 9 V + | | (9%) | | (14%) | | (8%) |
| Collection | | | | | | |
| Head | | | | | | |

TABLE 2b
Concentrations of hormones and glucose in
transdermal exudate collected by different
formats from sheep

| | |
|---|---|
| Insulin | Cortisol |

| | Serum, | Exudate, | Serum, | Exudate, |
|---|---|---|---|---|
| | ng/ml | ng/ml | ng/ml | ng/ml |
| SLS + UG | 0.2 ± 0.6 | ND | 10.9 ± 1.6 | ND |
| SLS + 10% E | 0.18 ± 0.09 | ND | 14.3 ± 2.8 | ND |
| SLS + 10% E + US | 0.4 ± 0.15 | 0.0004 ± 0.002 | 11.7 ± 2.4 | 0.55 ± 0.13 |
| 1 min | (1%) | (1%) | | (5%) |
| SLS + 10% E + US | 0.21 ± 0.08 | 0.0064 ± 0.0041 | 12.5 ± 2.8 | 1.1 ± 1.0 |
| 1 min + 9 V | | (3%) | | (9%) |
| SLS + 10% E + US | 0.19 ± 0.11 | 0.013 ± 0.006 | 13.1 ± 2.9 | 1.9 ± 0.93 |
| 1 min + 9 V + Collection | | (7%) | | (14%) |
| Head | | | | |

SLS: sodium lauryl sulfate,
UG: ultrasound gel,
E: ethanol,
US: ultrasound,
9-V electric field,
ND: not detectable.
Means ± S.E. are displayed.
(%) Relative recovery rounded to the nearest whole number, except for glucose exudate collected without ultrasound.

Immunosensors on-line with the collection head gave hormonal measurements that are strongly correlated (r²≧0.94) with the off-line analysis of effluent. However, the immunosensors were able to detect the hormonal concentrations in a smaller volume of sample fluid than when using off-line analysis (150 μl perfusion of chamber compared to 300 μl needed for effluent detection). Correspondingly, this meant that a smaller time span of each sample collection could be used (1.5 min versus 2 min). For the course of the experiment, however, to ensure maximum recovery, 300 μl (2 min) was used.

During the familiarization period, salivary cortisol values fell from 6.4±1.2 ng/ml on the first day to 1.1±0.9 ng/ml on Day 14. By Day 14, animals showed no subjective aversion to penning and handling.

On experimental days, the animals did, however, show some mild flinching behaviour with the jugular catheterisation, but little response to saliva collection, and no obvious response to the electrosonophoresis. In the case of jugular catheterisation, data (not shown) suggested that with 50 min of rest following the catheterisation, any hormone level change associated with this procedure had subsided to baseline.

Cortisol, 17-β estradiol, testosterone and glucose were all measurable in saliva. Insulin was not detectable. Prior to the exercise stress, all values showed a high correlation (all (r²≧0.67) with blood serum values (both free and total for hormones). This was best achieved when the saliva sample was compared to the blood sample obtained 20-30 min previously (i.e. there was a time lag between blood values and associated changes in saliva values). At rest, the levels of the hormones and glucose detected in saliva were typically between 4% and 9% of those seen in the serum. During and subsequent to exercise stress, cortisol, testosterone and glucose increased, in all animals, in both saliva and blood (FIG. 3 and Tables 3a and 3b). Insulin decreased in blood samples over the stress. However, the values in saliva and blood did not correlate as well as when the animal was at rest, irrespective of allowing any time lag between blood values and reflective saliva values. This reduction in correlation included 17-β estradiol even though it showed little change in either blood or saliva across the stress. FIG. 3 and Tables 3a and 3b illustrate this data.

TABLE 3a
Concentration of hormones and glucose in serum, transdermal
and saliva samples collected from sheep before, during and after exercise.

| | Glucose | | | Insulin | | |
|---|---|---|---|---|---|---|
| | Serum, | Saliva, | Exudate, | Serum, | Saliva, | Exudate, |
| | mg/l | mg/l | mg/l | ng/l | ng/l | ng/l |
| Preexercise | 734.8 ± 89.7 | 31.7 ± 3.9 | 70.5 ± 91.3 | 0.39 ± 0.08 | ND | 0.029 ± 0.009 |
| | | (0.76) (4%) | (0.87) (9%) | | | (0.79)(7.5%) |
| 10 min | 911.3 ± 96.8 | 32.9 ± 7.3 | 77.9 ± 11.6 | 0.21 ± 0.07 | ND | 0.016 ± 0.005 |
| Exercise | | (0.52)(3.5%) | (0.85)(8.5%) | | | (0.81)(7.5%) |
| 20 min | 1205.6 ± 101.3 | 34.5 ± 7.4 | 107.4 ± 14.6 | 0.12 ± 0.05 | ND | 0.009 ± 0.003 |
| Exercise | | (0.47)(3.0%) | (0.88)(9%) | | | (0.80)(7.5%) |
| 30 min | 1458.1 ± 112.7 | 36.0 ± 9.1 | 130.3 ± 13.4 | 0.11 ± 0.05 | ND | 0.008 ± 0.004 |
| Exercise | | (0.55)(2.5%) | (0.85)(9%) | | | (0.79)(7.0%) |
| Postexercise | 1179.8 ± 105.8 | 32.3 ± 8.41 | 102.6 ± 14.3 | 0.19 ± 0.08 | ND | 0.014 ± 0.008 |
| | | (0.67)(2.5%) | (0.86)(9%) | | | (0.83)(7.5%) |

TABLE 3b
Concentration of hormones and glucose in serum, transdermal and
saliva samples from sheep collected before, during and after exercise.

| | Estradiol | | | Cortisol | | |
|---|---|---|---|---|---|---|
| | Serum, | Saliva, | Exudate, | Serum, | Saliva, | Exudate, |
| | mg/l | mg/l | mg/l | ng/l | ng/l | ng/l |
| Preexercise | 1.4 ± 1.2 | 0.05 ± 0.03 | 0.12 ± 0.06 | 11.9 ± 4.7 | 1.07 ± 1.03 | 1.71 ± 0.85 |
| | | (0.67)(5%) | (0.82)(9%) | | (0.81)(9%) | (0.91)(14.5%) |
| 10 min | 1.5 ± 1.1 | 0.038 ± 0.011 | 0.14 ± 0.08 | 19.6 ± 5.1 | 1.51 ± 0.97 | 2.91 ± 0.83 |
| Exercise | | (0.49)(2.5%) | (0.79)(9%) | | (0.61)(8%) | (0.89)(15%) |
| 20 min | 1.3 ± 1.1 | 0.046 ± 0.009 | 0.12 ± 0.07 | 27.8 ± 4.6 | 2.35 ± 2.19 | 3.92 ± 1.01 |
| Exercise | | (0.45)(3.5%) | (0.80)(9%) | | (0.55)(8.5%) | (0.88)(14%) |
| 30 min | 1.5 ± 1.3 | 0.03 ± 0.012 | 0.14 ± 0.09 | 39.3 ± 7.4 | 2.44 ± 1.76 | 5.74 ± 0.96 |
| Exercise | | (0.51)(2%) | (0.82)(9%) | | (0.62)(6%) | (0.90)(15%) |
| Postexercise | 2.2 ± 1.1 | 0.052 ± 0.021 | 0.20 ± 0.06 | 18.3 ± 4.9 | 1.34 ± 1.81 | 2.71 ± 0.55 |
| | | (0.59)(4%) | (0.79)(9%) | | (0.72)(7%) | (0.89)(15%) |

Means ± S.E. are displayed.
( ) Indicates r² value for that period.
(%) Relative recovery rounded to the nearest 0.5% based on mean values.
Preexercise, exercise and postexercise values for transdermal and blood samples represent pooled.
Measurements for the periods are indicated.
For saliva samples, preexercise includes saliva samples at 10 and 20 min into exercise, exercise values for saliva samples are 30, 40 and 50 min from the start of exercise.
This allows a 20-min lag between values in the blood and their reflection in saliva.

Salivary values measured directly by off-line assay or in real time via the immunosensors were highly correlated (r²≧0.92) for all values across the experiment.

In transdermal exudates, cortisol, 17-β estradiol, testosterone, insulin and glucose were all measurable. Exudate values were typically between 9% and 15% of blood values in sheep, varying with both the measured hormone and the individual. Despite considerable interindividual variation, within an individual, the relationship between exudate and blood was consistent, for each hormone, before, across and after the exercise stress. Within individual animals, a strong correlation (r²≧0.79) was seen between the exudate values and blood values obtained at the same time (i.e. there was no lag between blood levels and reflective transdermal levels, provided 10 min was allowed after the initial application of ultrasound). To maintain this transdermal flux, ultrasound is needed to be applied at least every 30 min. Cortisol, testosterone and glucose rose significantly (P≦0.05) with the exercise, while insulin decreased (P≦0.05) and 17-β estradiol showed little change. Tables 3a and 3b and FIG. 3 exhibit this data.

Measurements did not change significantly across the three repeat exercise stressors for transdermal exudates and blood values. Baseline saliva values also showed stability, but saliva values across exercise and recovery were also variable (P≦0.05) across the repeats.

### EXAMPLE 5

### Human Tests

Ten human volunteers (males aged 19-23, body weights 85-103 kg) were seated at 09:30 h. During the experiment, they were held at a room temperature of 20-22° C. An initial 1 min burst of ultrasound, as for the sheep (above), was made on the volar surface of the forearm, following application of SLS-EG mix. Ten minutes later, transdermal (electrosonophoresis) collection began from this site on this forearm, simultaneous to a venepuncture blood collection being made from the other. A saliva collection was then made, followed by a second, third and fourth saliva collection 20, 30 and 40 min after the blood sample. These measurements were repeated three times over 3 h (i.e. a total of three blood and transdermal samples and 12 saliva samples). In between each sampling series, subjects were allowed to walk around, eat lightly and drink water. Following this collection, the five volunteers exercised at moderate intensities, for 40 min (heart rates maintained in the range of 130-160 bpm) on a rowing machine, stopping briefly at 10, 20 and 30 min to give blood, transdermal and saliva samples. The subjects gave another set of samples at 40 min upon completion of rowing, then warmed down lightly with stretching exercises for a further 10 min and gave a further saliva sample at the end of this time. Two more saliva samples were collected at 60 and 70 min after exercise started. At the end of this time, sampling was repeated as above, prior to exercise, for a further 3 h. The entire procedure was repeated twice a week for 3 weeks (a total of six times). The distance rowed (m) represented the total work rate during the exercise.

In a separate series of experiments on these human subjects, the transdermal and blood samplings were repeated as for the nonexercising period, however, on each transdermal sample, a different site on the volar forearm was used, ranging from wrist to elbow level. This was then repeated with the room temperature increased to 28-30° C.

Cortisol, testosterone, 17-β estradiol and glucose were detected in saliva while insulin was not. Correlation to blood values preexercise was high (r²≧0.69) when the salivary values were compared to blood values 30 min prior to the saliva collection (i.e. a time lag of 30 min). During exercise and recovery, correlation between saliva and blood for all values fell (r²≧0.42) considerably irrespective of any time lag considerations. Salivary values, at rest, were typically 3-10% of blood values, depending on the hormone. Salivary values from the same samples, measured directly by off-line assay or in real time via the immunosensors, were highly correlated (r²≧0.91) for all values across the experiment.

Transdermal exudates contained measurable quantities of cortisol, testosterone, 17-β estradiol, insulin and glucose. After the initial application of ultrasound (allowing 10 min), these correlated (r²≧0.82) with blood values obtained at a similar time (i.e. no time lag). This was not changed across exercise and recovery.

Exudate values were between 8% and 12%, depending on the hormone, of blood values. This value for each hormone was consistent within subjects and across repeat measures. Intersubject variation was also considerably less (P≦0.05) than seen in sheep.

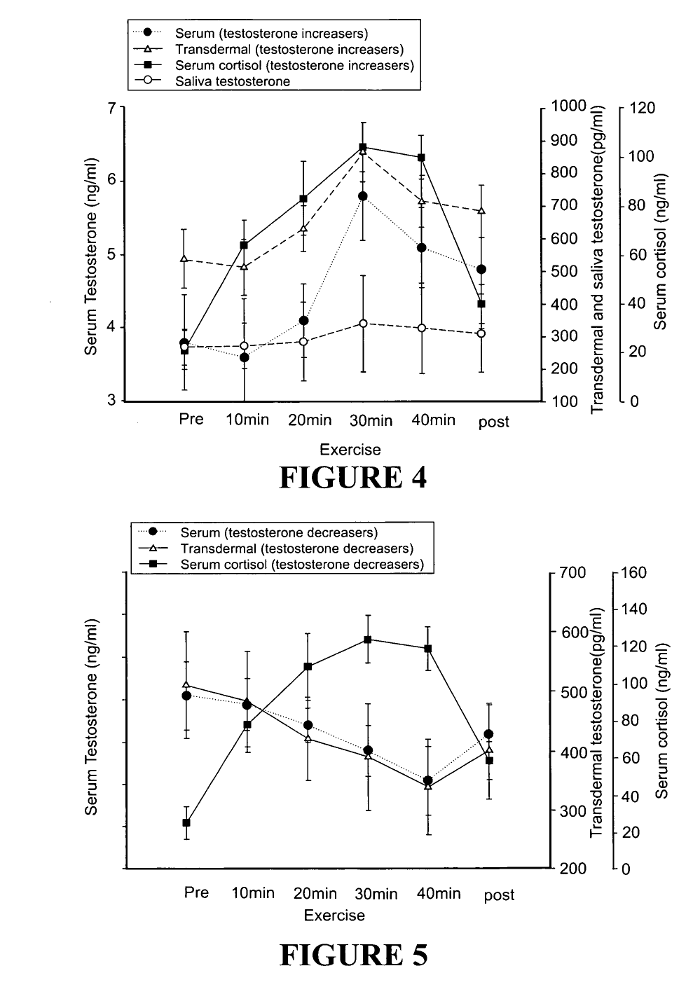

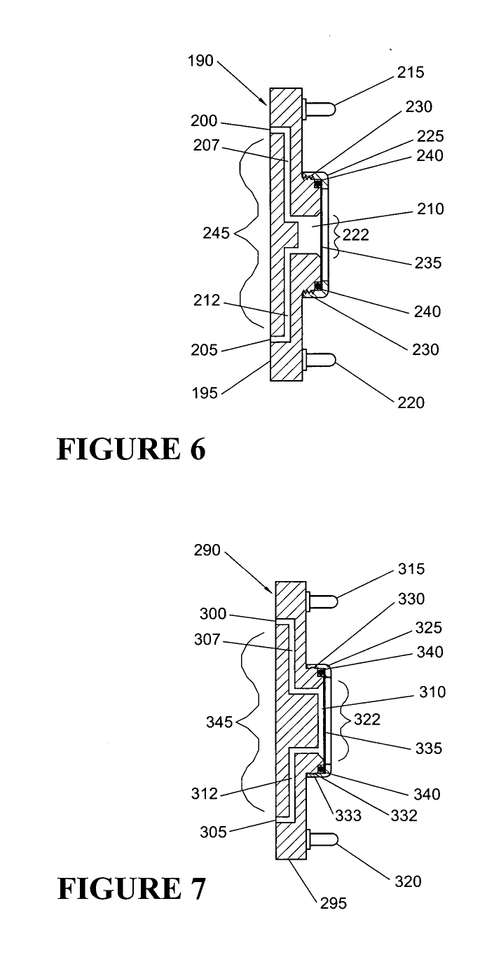

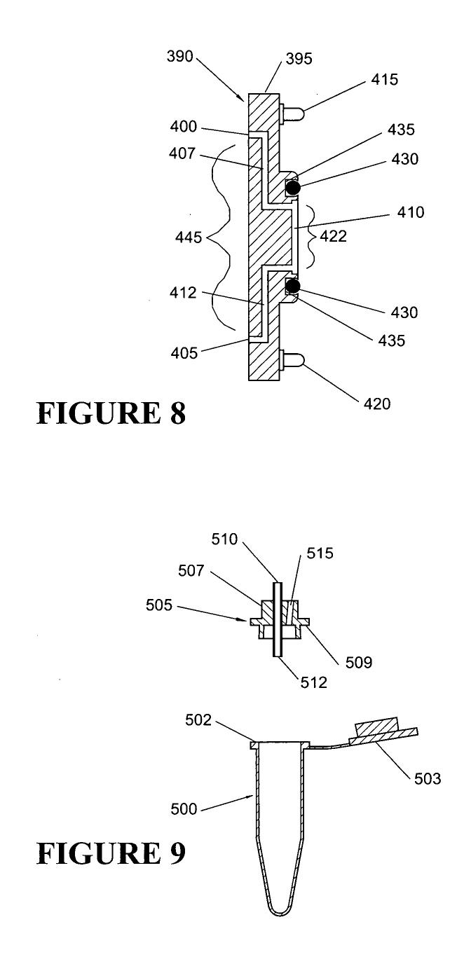

In all subjects, cortisol increased (P≦0.05) with exercise, but insulin decreased (P≦0.05). Testosterone and 17-β estradiol were more variable, showing individual subject-related increases or decreases across exercise and recovery. Glucose increased (P≦0.05) across exercise and recovery. Tables 4a and 4b and FIGS. 4 and 5 display this data. Over the six exercise sessions, the type and magnitude of the stressor response did not significantly differ for individuals.

TABLE 4a
Concentration of hormones and glucose in serum, transdermal and
saliva samples collected from human subjects before, during and after exercise.

| | Glucose | | | Insulin | | |
|---|---|---|---|---|---|---|
| | Serum, | Saliva, | Exudate, | Serum, | Saliva, | Exudate, |
| | mg/l | mg/l | mg/l | μIU/ml | μIU/ml | μIU/ml |
| Preexercise | 814.9 ± 112.6 | 28.5 ± 7.9 | 79.0 ± 13.4 | 16.3 ± 5.4 | ND | 1.35 ± 0.90 |
| | | (0.78)(3.5%) | (0.87)(10%) | | | (0.83)(8%) |
| 0 min | 905.4 ± 89.7 | 31.3 ± 9.2 | 86.1 ± 9.3 | 13.1 ± 3.0 | ND | 1.05 ± 0.45 |
| Exercise | | (0.51)(3.5%) | (0.88)(10%) | | | (0.82)(8%) |
| 10 min | 119.3 ± 106.7 | 33.6 ± 6.5 | 110.8 ± 12.7 | 10.6 ± 4.9 | ND | 0.90 ± 0.28 |
| Exercise | | (0.55)(3%) | (0.84)(10%) | | | (0.84)(8.5%) |
| 20 min | 1205.8 ± 114.3 | 34.3 ± 7.1 | 118.1 ± 17.4 | 6.3 ± 3.8 | ND | 0.51 ± 0.23 |
| Exercise | | (0.54)(3%) | (0.86)(10%) | | | (0.83)(8%) |
| 30 min | 1509.7 ± 201.4 | 35.1 ± 8.3 | 143.4 ± 19.6 | 5.4 ± 4.1 | ND | 0.46 ± 0.21 |
| Exercise | | (0.61)(2%) | (0.85)(10%) | | | (0.83)(8.5%) |
| 40 min | 1519.3 ± 186.5 | 36.0 ± 7.5 | 147.5 ± 21.3 | 6.9 ± 3.8 | ND | 0.57 ± 0.33 |
| Exercise | | (0.69)(2%) | (0.88)(10%) | | | (0.80)(8%) |
| Postexercise | 1005.4 ± 103.7 | 32.1 ± 8.3 | 96.5 ± 19.2 | 14.1 ± 5.5 | ND | 1.15 ± 0.48 |
| | | (0.65)(3%) | (0.89)(10%) | | | (0.83)(8%) |

TABLE 4b
Concentration of hormones and glucose in serum, transdermal
and saliva samples collected from
human subjects before, during and after exercise.

| | Estradiol | | | Cortisol | | |
|---|---|---|---|---|---|---|
| | Serum, | Saliva, | Exudate, | Serum, | Saliva, | Exudate, |
| | ng/l | ng/l | ng/l | ng/l | ng/l | ng/l |
| Preexercise | 15.6 ± 5.7 | 0.71 ± 0.25 | 1.23 ± 0.46 | 22.7 ± 8.1 | 2.25 ± 1.04 | 2.79 ± 0.85 |
| | | (0.69)(4.5%) | (0.82)(8%) | | (0.89)(10%) | (0.93)(12%) |
| 0 min | 19.1 ± 6.3 | 0.76 ± 0.38 | 1.53 ± 0.59 | 29.5 ± 9.2 | 2.79 ± 1.13 | 3.69 ± 1.31 |
| Exercise | | (0.42)(4%) | (0.85)(8.0%) | | (0.76)(9.5%) | (0.91)(12.5%) |
| 10 min | 18.3 ± 7.1 | 0.75 ± 0.51 | 1.47 ± 0.94 | 7.08 ± 11.5 | 8.01 ± 2.86 | 8.57 ± 1.94 |
| Exercise | | (0.44)(4%) | (0.88)(8.0%) | | (0.84)(11%) | (0.89)(12%) |
| 20 min | 21.5 ± 7.5 | 0.83 ± 0.39 | 1.74 ± 0.54 | 91.3 ± 19.7 | 8.75 ± 4.31 | 11.5 ± 2.8 |
| Exercise | | (0.55)(4%) | (0.82)(8%) | | (0.71)(9.5%) | (0.91)(12.5%) |
| 30 min | 26.7 ± 9.2 | 0.85 ± 0.22 | 2.14 ± 0.86 | 115.7 ± 15.8 | 10.04 ± 3.11 | 14.4 ± 3.7 |
| Exercise | | (0.42)(3%) | (0.84)(8.0%) | | (0.79)(9.0%) | (0.92)(12.5%) |
| 40 min | 19.4 ± 4.0 | 0.68 ± 0.26 | 1.53 ± 0.51 | 109.4 ± 13.1 | 9.19 ± 3.76 | 13.7 ± 2.4 |
| Exercise | | (0.68)(3.5%) | (0.84)(8%) | | (0.85)(8.5%) | (0.90)(12.5%) |
| Postexercise | 13.9 ± 5.1 | 0.45 ± 0. | 1.11 ± 0.55 | 49.6 ± 11.3 | 4.02 ± 1.95 | 6.07 ± 1.14 |
| | | (0.53)(3%) | (0.83)(8.0%) | | (0.81)(8%) | (0.88)(12%) |

Means ± S.E. are displayed.
( ) Indicates r² value for that period.
(%) Relative recovery rounded to the nearest 0.5% based on mean values.
Preexercise, exercise and postexercise values for transdermal and blood samples represent pooled measurements for the periods indicated.
For saliva samples, preexercise includes saliva samples at 10, 20 and 30 min into exercise, exercise values for saliva are 40, 50, 60 and 70 min from start of exercise.
This allows a 30-min lag between values in the blood and their reflection in saliva.

In all subjects, cortisol increased significantly (P≦0.05) with exercise. In some subjects, during exercise, testosterone levels fell, while in other subjects, testosterone increased. This was a consistent individual pattern over the six exercise repeats. In subjects where testosterone increased over exercise, an increase in distance rowed in the exercise time was seen (P≦0.05) progressively over the exercise repeats. When the subjects were questioned, they did not report any information that suggested differences in terms of other fitness work, sleep or diet.

Placement of sampling device at different sites of the volar forearm did not significantly affect recovery or measurement within subjects. Increasing the room temperature by 8-10 ° C. tended to increase recovery slightly by 1-3%, but this was not significant.

### EXAMPLE 6

### Flow Rate Effect on Analyte Extraction

Using the methods above for transudate and serum extraction, an experiment was conducted to determine the effect of flow rates through the head on testosterone recovery. Table 5 sets out the results of the experiment.

In a series of experiments (15 measures per flow rate) flow rates were compared for collection of testosterone in exudate from electrosonophoresis compared to that concurrently measured in serum of the subjects.

TABLE 5

| | | Collected | | |
|---|---|---|---|---|
| | Sonified off | in | | |
| | membrane* | exudate | Serum level at | Number of |
| Flow rate | (ng/ml) | (ng/ml) | same time (ng/ml) | samples tested |
| 50 μl/min | 0.1-0.2 | 0.05-0.09 | 1.9-2.5 | 15 |
| 100 μl/min | 0.05-0.07 | 0.15-0.20 | 2.0-2.4 | 15 |
| 150 μl/min | 0.01-0.02 | 0.2-0.3 | 1.9-2.6 | 15 |
| 200 μl/min | 0.01-0.02 | 0.21-0.30 | 2.0-3.01 | 15 |
| 300 μl/min | 0.08-0.12 | 0.12-0.17 | 1.9-2.8 | 15 |

*Membrane removed and subject to sonication in water bath. Amount of analyte released measured.

Increasing flow rate through a constant volume chamber was inversely propotional to the amount of testosterone sonified off the membrane had a marked effect on collection versus amount trapped on membrane. The lower the flow rate, the less exudate was collected. The exception to this was at 300 μl/min, where the maximum transfer rate across the biological membrane for the analyte appears to have been reached. The increased flow caused dilution of the transudate.

### EXAMPLE 7

### Flow Rate Optimisation for Different Analytes

A similar experiment to that in example 10 was conducted on cortisol. The results are set out in Table 6.

TABLE 6

| | Sonified off | Collected in | Serum level | |
|---|---|---|---|---|
| | membrane* | exudate | at same | Number of |
| Flow rate | (ng/ml) | (ng/ml) | time (ng/ml) | samples tested |
| 50 μl/min | 1.2-3.4 | 0.5-2.3 | 19-55 | 10 |
| 100 μl/min | 1.1-3.1 | 0.8-1.6 | 20-44 | 10 |
| 150 μl/min | 0.5-1.1 | 1.2-4.3 | 19-59 | 10 |
| 200 μl/min | 0.2-0.7 | 1.4-2.7 | 20-33 | 10 |
| 300 μl/min | 0.1-0.4 | 1.5-5.9 | 19-68 | 10 |

### EXAMPLE 8

### Other Analytes

1. Caffeine

TABLE 7

| | Transdermal | |
|---|---|---|
| Number of subjects | collection levels | Serum levels |
| 10 | 0.2-0.8 mg/ml (r = 0.89) | 1-5 mg/ml |
| 10 | 0.3-1.5 mg/ml | 4-14 mg/ml (loading) |
| | (r = 0.91) | |

2. Ethanol

TABLE 8

| | Transdermal | |
|---|---|---|
| Number of subjects | collection levels | Serum levels |
| 5 | 0.02-0.08 mg/ml | 0.01-0.9 mg/ml |
| | (r = 0.85) | |
| 5 | 0.05-0.11 mg/ml | 0.4-1.4 mg/ml (loading) |
| | (r = 0.87) | |

3. Progesterone

TABLE 9

| | Transdermal | |
|---|---|---|
| Number of subjects | collection levels | Serum levels |
| 3 | 0.2-1.1 ng/ml (r = 0.88) | 1-12 ng/ml |
| 5 | 0.5-2.8 ng/ml | 8-25 ng/ml |
| | (r = 0.91) | |

4. Human Chorionic Gonadotropin

TABLE 10

| Number of | Transdermal | |
|---|---|---|
| subjects | collection levels | Serum levels |
| 10 | 0.002-0.08 mIU/ml (r = 0.75) | 0.01-0.9 mIU/ml |
| 10 | 0.15-11 mIU/ml (r = 0.84) | 1.5-100 mIU/ml females |
| | | pregnant |

5. Prolactin

TABLE 11

| | Transdermal | |
|---|---|---|
| Number of subjects | collection levels | Serum levels |
| 10 | 0.02-0.8 ng/ml (r = 0.79) | 1-8 ng/ml |
| 5 | 0.4-10 ng/ml | 4-100 ng/ml females |
| | (r = 0.92) | pregnant and lactating |

6. Procalcitonin/TNF Alpha and IL 6 (Measure of Bacterial Sepsis)

TABLE 12

| | | Procalcitonin | TNF alpha | TNF alpha | IL 6 | IL 6 |
|---|---|---|---|---|---|---|
| Subject | Procalcitonin | Transdermal | Trandermal | Transdermal | Serum | Transdermal |
| number | Serum ng/ml | ng/ml | ng/ml | ng/ml | ng/ml | ng/ml |
| 30 | 0.1-0.5 | 0.02-0.05 | 0.1-0.5 | 0.005-0.01 | 0.1-0.3 | 0.03-0.05 |
| | | r = 0.85 | | r = 0.79 | | r = 0.81 |
| 20 | 0.5-15 | 0.04-1.7 | 0.5-12 | 0.03-1.4 | 0.3-11.5 | 0.04-1.2 |
| | | r = 0.88 | | r = 0.91 | | r = 0.87 |

7. Patients Recovering in Intensive Care—Levels of an Anaesthetic Propofol and Cortisol

TABLE 13

| | | | | | Propofol | | | Cortisol |
|---|---|---|---|---|---|---|---|---|
| | | | | | (ppm) | | | (ng/ml) |
| Time | | Sample/Patient | | Sex | Blood | US | Blood | US |
| 13- | 16:30 | E1 | DLX3519 | F | 1.54 | 0.69 | 104.6 | 36.4 |
| Nov | 17:45 | E2 | DLX3520 | F | 0.85 | 0.39 | 91.8 | 31.3 |
| 14- | 14:00 | F1 | DUV5231 | M | 1.41 | 0.65 | 127.8 | 45.9 |
| Nov | | | | | | | 27 | |
| | 15:30 | F2 | DUV5232 | M | 0.91 | 0.29 | 84.5 | 32.1 |
| | 18:10 | G1 | EJV2613 | F | 0.94 | 0.31 | 110.4 | 37.4 |
| | 19:20 | G2 | EJV2614 | F | 0.21 | 0.1 | 84.8 | 30.5 |
| 16- | 13:40 | I1 | AMX8522 | F | 1.63 | 0.74 | 109.5 | 39.7 |
| Nov | 14:55 | I2 | AMX8523 | F | 0.79 | 0.36 | 76.9 | 29.2 |

8. Pseudoephedrine

TABLE 14

| | Transdermal | |
|---|---|---|
| Number of subjects | collection levels | Serum levels |
| 5 | 0.2-0.8 μg/ml (r = 0.83) | 1-8 μg/ml |
| 5 | 0.4-2.1 μg/ml | 4-25 μg/ml females |
| | (r = 0.91) | pregnant and lactating |

### EXAMPLE 9

### Effect of Flow Profile

Two different collecting chambers were fashioned. One chamber had a rectangular profile in cross section, giving a cuboid shaped chamber. The other chamber had a circular profile, giving a cylindrical shaped chamber with a rounded cross section when viewed from the test site.

Both chambers form part of a sample head (50) as illustrated in FIG. 2. Both chambers were machined to have a volume of 300 μl. Flow rates through the chamber were set at 300 μl/min. A glucose solution was placed on the other side of the membrane (105). The relative recoveries of glucose were determined.

The rectangular profile chamber provided a recovery of 5.2+/−1.2% of the glucose (based on 10 measures)

The circular profile chamber had a recovery of 9.8% +/−2.0 (based on 30 measures).

### EXAMPLE 10

### Effect of Flow Profile

A sample head as illustrated in FIG. 2 having a circular profile of 300 μl chamber flow and using a flow rate of 300 μl/min was employed. An inert shapable putty was introduced into the chamber and pushed down flat to cover approx the bottom 0.5-0.8 mm of the chamber. In a glucose recovery experiment on this chamber, 5 measures recovered 8.3+/−1.4% of the glucose solution.

The same chamber was employed with the same piece of putty. However, the putty was shaped into a thin column in middle of chamber extending up to, but not touching, the membrane in a glucose recovery experiment, 4.5+/−1.6% of the glucose solution was recovered.

### Discussion

Transdermally collected exudate appeared to mirror blood changes with high consistency. Collection was made from subjects with relative ease and in human subjects without any report of pain or discomfort. Animals showed no obvious behavioural aversion to the procedure, and had been familiarized to the overall experiment procedures.

The device and method of the invention have high potential for measuring the performance of athletes. In human subjects, exercise-related increases were predictive of the subject's ability to increase work performance, as measured in distance rowed, on a subsequent training session. Increases in cortisol relative to testosterone, at rest, have been suggested as indicative of over-training in human subjects, with a fall in testosterone being partly contributory. The invention may therefore be useful in in determining training performance in athletes.

The device and method of the invention are very useful for non-invasive sampling. The device is portable and handy, more successful in prediction of blood levels than saliva over varied conditions and easy to use in animal studies. With short sampling time and intensive longitudinal studies, it has advantages over attempts at repeated saliva collection. Combining immunosensors with sample collection offers the additional benefit of rapid measurement on-line. No evidence of skin damage or pain, or of aversion, was seen with repeat measurement. There are numerous benefits that the invention offers human and other animal endocrine studies, including addressing welfare and stress concerns surrounding invasive sampling.

It will be appreciated by those persons skilled in the art that the above description is provided by way of example only and that the invention is not limited thereto.

1. A transudate extraction device for non-invasive extraction of transudate through a target area of a skin barrier of an organism subjected to sufficient ultrasonic energy to induce transudation therethrough, the device including at least:
a contacting means for contacting said target area to receive transudate therefrom,
a transudate collection chamber in open communication with said contacting means to receive transudate therefrom,
circulating means for circulating transudate within said collection chamber to thereby create and maintain a concentration gradient through the skin of a said test subject, said collection chamber having a discharge opening through which exudate collected therein can be discharged.

2. A device according to claim 1 wherein said contacting means and said collection chamber are incorporated into a common housing.

3. A device according to claim 1 or claim 2 wherein said contacting means is a transudate permeable or semi-permeable membrane.

4. A device according to claim 1 or claim 2 wherein said contacting means is an O-ring.

5. A device according to any one of the preceding claims wherein said collection chamber is of a regular geometric shape in cross-section.

6. A device according to claim 5 wherein said collection chamber is circular in cross section.

7. A device according to any one of the preceding claims wherein said circulating means comprises a flow of liquid into and out of said collection chamber.

8. A device according to claim 7 wherein there are provided an inlet conduit and an outlet conduit through which said flow of liquid is maintained, a source of liquid communicating with said inlet conduit and a collecting means communicating with said outlet conduit.

9. A device according to claim 8 wherein said collecting means is a collecting vessel.

10. A device according to claim 9 wherein said collecting vessel is a test-tube.

11. A device according to any one of the preceding claims which includes an analysing means for analysing the composition of said transudate.

12. A device according to claim 11 wherein said analysing means is in said collection chamber.

13. A device according to claim 12 wherein said analysing means is in said outlet conduit.

14. A device according to any one of claims 11 to 13 wherein said analysing means is capable of detecting an analytes in said transudate.

15. A device according to any one of the preceding claims including means for providing an electric charge across said target area, associated with said contacting means.

16. A device according to any one of the preceding claims having an ultrasonic generator attaching means, said attaching means being adapted to orient a said ultrasonic generator therein to direct ultrasonic energy onto said target area.

17. A method for non-invasively extracting transudate through a skin barrier of an organism, which includes at least the steps of:
treating a target area of a said skin barrier with a sufficient concentration of ultrasonic energy to induce transudation therethrough;
collecting said transudate thereby produced in a dynamic flow of fluid, said dynamic flow being controlled so as to provide a concentration gradient in a direction which induces continuing transudation through said skin barrier, and recovering transudate from said dynamic flow.

18. A method according to claim 17 wherein the frequency of said ultrasound energy is in the range 1-3 MHz.

19. A method according to claim 17 wherein the frequency of said ultrasound energy is in the range 10-30 kHz.

20. A method according to any one of claims 17 to 19 wherein the output of said ultrasound energy is in the range 0.1 to 3 W/cm².

21. A method according to any one of claims 17 to 19 wherein the output of said ultrasound energy is in the range 5-15 W/cm².

22. A method according to any one of claims 17 to 21 wherein said fluid is saline.

23. A method according to any one of claims 17 to 22 wherein said ultrasound energy is pulsed on and off.

24. A method according to claim 23 wherein said ultrasound energy is pulsed for about 5 seconds on and 5 seconds off.

25. A method according to any one of claims 17 to 24 wherein said transudate is passed through a permeable or semi permeable membrane interposed between said skin barrier and said dynamic flow of liquid.

26. A method according to any one of claims 17 to 25 wherein the method further includes the step of collecting and storing transudate in a collection vessel.

27. A method according to any one of claims 17 to 26 which includes the additional step of analysing said transudate.

28. A method according to claim 27 wherein said analysing step comprises detecting one or more analytes in the transudate.

29. A method according to claim 27 or claim 28 wherein said analysing step is carried out substantially contemporaneously with the extraction of transudate.

30. A method according to any one of claims 17 to 29 wherein turbulence in the fluid stream is minimised.

31. A method according to any one of claims 17 to 30 wherein flow rate of said dynamic flow of fluid is in the range of 50 to 600 μl/min.

32. A method according to any one of claims 17 to 31 that additionally includes enhancing extraction of an analyte through the skin before or contemporaneously with extracting transudate.

33. A method according to claim 32 wherein enhancing extraction of the analyte is achieved by subjecting said skin barrier to an electromagnetic field.

34. A method according to any one of claims 17 to 33 that additionally includes the step of sanitising the fluid stream.

35. A replaceable member adapted for attachment to the test area of a device of any one of claims 1 to 16.
